(19) United States
(12) Patent Application Publication
(10) Pub. No.: US 2026/0078364 A1
(43) Pub. Date: Mar. 19, 2026

(54) DEVICES AND SYSTEMS FOR DNA CAPTURE

(71) Applicant: The Johns Hopkins University, Baltimore, MD (US)

(72) Inventor: Bradley Downs, Baltimore, MD (US)

(21) Appl. No.: 19/115,078

(22) PCT Filed: Sep. 26, 2023
(86) PCT No.: PCT/US2023/033672

§ 371 Date: Mar. 25, 2025

Related U.S. Application Data
(60) Provisional application No. 63/377,154, filed on Sep. 26, 2022.

(51) Int. Cl. C12N 15/10; C12N 9/22; C12N 11/00; C12N 15/113; C12Q 1/6834; C12Q 1/6844; C12Q 1/6869

(52) U.S. Cl. CPC C12N 15/1003; C12N 9/226; C12N 11/00; C12N 15/113; C12Q 1/6834; C12Q 1/6844; C12Q 1/6869; C12N 2310/20

(57) ABSTRACT

The present disclosure provides devices, systems, kits, and related methods for isolating DNA from a sample (e.g., plasma) using RNA-guided DNA binding proteins (e.g., Cas proteins). Particularly, the disclosure provides devices, systems, kits, and related methods for Cas9 mediated capture of circulating free DNA (cfDNA) from flowing plasma.

### CROSS-REFERENCE TO RELATED APPLICATIONS

This application claims the benefit of U.S. Provisional Application No. 63/377,154, filed Sep. 26, 2022, the content of which is herein incorporated by reference in its entirety.

### STATEMENT REGARDING FEDERALLY SPONSORED RESEARCH OR DEVELOPMENT

This invention was made with government support under grant number W81XWH1810482 awarded by the Department of Defense. The government has certain rights in the invention.

### FIELD

The present disclosure provides devices and systems suitable for capturing or isolating DNA (e.g., circulating free DNA (cfDNA)) from a sample (e.g., plasma).

### BACKGROUND

Noninvasive detection of circulating tumor DNA (ctDNA) in blood-based liquid biopsies can be used to detect stages of cancer, track tumor burden, and monitor response to treatment.

However, a limiting factor in using blood-based liquid biopsies for cancer detection is the volume of plasma required to capture a measurable number of ctDNA copies.

### SUMMARY

Disclosed herein are devices and systems. In some embodiments, the systems and devices are suitable for capturing DNA from a sample.

In some embodiments, the devices comprise: a sample inlet; a sample outlet; a channel fluidically connecting the sample inlet and sample outlet, wherein the channel comprises at least one mixing element; and a plurality of capture complexes each capture complex comprising an RNA-guided DNA binding protein, or a functional fragment thereof, bound to a guide RNA configured to at least partially hybridize to a circulating free DNA (cfDNA) of interest. In some embodiments, the capture complexes are immobilized on one or more interior surfaces of the channel.

In some embodiments, the channel is substantially linear.

In some embodiments, the at least one mixing element comprises one or more chambers spanning the width of the channel and expanding out from the channel. In some embodiments, the one or more chambers expand out perpendicularly from direction of the channel. In some embodiments, the capture complexes are immobilized on one or more interior surfaces of the one or more chambers.

In some embodiments, the at least one mixing element comprises a plurality of microstructures. In some embodiments, the microstructures include ridges, channels, protrusions, or any combination thereof.

In some embodiments, the at least one mixing element has a height of between 30-80% of height of the channel.

In some embodiments, the plurality of capture complexes are linked to magnetic particles. In some embodiments, the plurality of capture complexes are linked by a biotin streptavidin linker. In some embodiments, the RNA-guided DNA binding protein further comprises a biotin tag. In some embodiments, the plurality of capture complexes are magnetically immobilized to the one or more locations on the interior surfaces of the channel. In some embodiments, the device further comprises one or more magnets.

In some embodiments, the plurality of capture complexes are immobilized through immunoprecipitation.

In some embodiments, the RNA-guided DNA binding protein is a CRISPR-associated (Cas) protein. In some embodiments, the RNA-guided DNA binding protein is Cas9. In some embodiments, the Cas9 is catalytically inactivated.

In some embodiments, the plurality of cfDNA complexes comprises more than one type of cfDNA complex each type having a guide RNA configured to at least partially hybridize to a different cfDNA of interest.

In some embodiments, the systems comprise: a plurality of RNA-guided DNA binding proteins, or a functional fragment thereof; one or more guide RNAs configured to at least partially hybridize to a cfDNA of interest; and a device comprising: a sample inlet; a sample outlet; and a channel fluidically connecting the sample inlet and sample outlet, wherein the channel comprises at least one mixing element, wherein the RNA-guided DNA binding proteins are configured for immobilization on one or more interior surfaces of the channel.

In some embodiments, the channel is substantially linear.

In some embodiments, the at least one mixing element comprises one or more chambers spanning the width of the channel and expanding out from the channel. In some embodiments, the one or more chambers expand out perpendicularly from direction of the channel. In some embodiments, the capture complexes are immobilized on one or more interior surfaces of the one or more chambers.

In some embodiments, the at least one mixing element comprises a plurality of microstructures. In some embodiments, the microstructures include ridges, channels, protrusions, or any combination thereof.

In some embodiments, the at least one mixing element has a height or depth of between 30-80% of height of the channel.

In some embodiments, the plurality of capture complexes are linked to magnetic particles. In some embodiments, the plurality of capture complexes are linked by a biotin streptavidin linker. In some embodiments, the RNA-guided DNA binding protein further comprises a biotin tag. In some embodiments, the plurality of capture complexes are magnetically immobilized to the one or more locations on the interior surfaces of the channel. In some embodiments, the device further comprises one or more magnets.

In some embodiments, the plurality of capture complexes are immobilized through immunoprecipitation.

In some embodiments, the RNA-guided DNA binding protein is a CRISPR-associated (Cas) protein. In some embodiments, the RNA-guided DNA binding protein is Cas9. In some embodiments, the Cas9 is catalytically inactivated.

In some embodiments, the one or more guide RNAs are bound to the plurality of RNA-guided DNA binding proteins to form a capture complex.

In some embodiments, the systems further comprise a sample. In some embodiments, the sample is a biological sample. In some embodiments, the biological sample comprises blood or blood components. In some embodiments, the blood component comprises plasma.

Further disclosed herein are methods for using the described devices and systems for capturing DNA from a sample. The methods comprise introducing the sample into a device as disclosed herein to form a complex comprising a capture complex and the circulating free DNA of interest. In some embodiments, the methods further comprise immobilizing the capture complexes in the device.

In some embodiments, the sample is introduced at a flow rate of 3-120 mL/min.

In some embodiments, the mixing element comprises two chambers along a single portion of the channel, perpendicular to sample flow direction in the channel, and the flow rate is 70-120 mL/min In some embodiments, the mixing element comprises two chambers along different portions of the channel, perpendicular to sample flow direction in the channel, and the flow rate is 10-30 mL/min.

In some embodiments, the mixing element comprises a plurality of microstructures and the flow rate is 10-20 mL/min.

In some embodiments, the sample is a biological sample. In some embodiments, the biological sample comprises blood or blood components. In some embodiments, the blood component comprises plasma.

In some embodiments, the circulating free DNA is of microbial or viral origin. In some embodiments, the circulating free DNA is circulating tumor DNA.

In some embodiments, the methods further comprise removing remaining sample from the device.

In some embodiments, the methods further comprise analyzing at least a portion of the sample or the remaining sample for the presence or absence of at least one biomarker.

In some embodiments, the methods further comprise returning at least a portion of the remaining sample to the subject.

In some embodiments, the methods further comprise one or more of: purifying the circulating free DNA of interest; amplifying the circulating free DNA of interest; and sequencing the circulating free DNA of interest.

Other aspects and embodiments of the disclosure will be apparent in light of the following detailed description and accompanying figures.

### BRIEF DESCRIPTION OF THE DRAWINGS

FIGS. 1A-1C show the development and investigation of the capture rate of ctDNA in plasma with a vortex-generating flow cell with a 0.5 mm wide flow channel and 18 mm wide capture chamber. FIG. 1A is an illustration and design of a vortex-generating flow cell. Dot plots showing the number of captured BRAF^{Mut} (FIG. 1B) and off-target ACTB DNA (FIG. 1C) copies after flowing 10 mL of plasma with 4,000 spiked in BRAF^{Mut} copies for 20 minutes at a range of flow rates between 6 and 104 mL/min; P=coefficient.

FIGS. 2A-2C show the development and investigation of the capture rate of ctDNA with a vortex-generating flow cell with a 5 mm wide flow channel and 18 mm wide capture chamber. FIG. 2A is an illustration and design of a vortex-generating flow cell. Dot plots showing the number of captured BRAF^{Mut} (FIG. 2B) and off-target ACTB DNA (FIG. 2C) copies after flowing 10 mL of plasma with 4,000 spiked in BRAF^{Mut} copies for 20 minutes at a range of flow rates; P=coefficient.

FIGS. 3A-3C show the development and investigation of the capture rate of ctDNA with a vortex-generating flow cell with two 9 mm wide capture chambers. FIG. 3A is an illustration and design of a vortex-generating flow cell. Dot plots showing the number of captured BRAF^{Mut} (FIG. 3B) and off-target ACTB DNA (FIG. 3C) copies after flowing 10 mL of plasma with 4,000 spiked-in BRAF^{Mut} copies for 20 minutes at a range of flow rates; P=coefficient.

FIGS. 4A-4C show the development and investigation of the optimal capture rate of ctDNA with a positive staggered herringbone mixer flow cell. FIG. 4A is an illustration and design of a herringbone mixer insert (left) and flow cell (right). Dot plots showing the number of captured BRAF^{Mut} (FIG. 4B) and off-target ACTB DNA (FIG. 4C) copies after flowing 10 mL of plasma with 4,000 spiked in BRAF^{Mut} copies for 20 minutes at a range of flow rates; P=coefficient.

FIGS. 5A-5D are dot plots showing the number, rate, and reproducibility of DNA copies captured over time using 10 mL of plasma with 4,000 spiked-in BRAF^{Mut} DNA copies. Number of BRAF^{Mut} copies (FIG. 5A) and off-target ACTB copies (FIG. 5B) captured using the vortex-generating flow cell with the flow channel width of 0.5 mm and capture chamber width of 18 mm at a flow rate of 78 mL/min. Number of BRAF^{Mut} copies (FIG. 5C) and off-target ACTB copies (FIG. 5D) captured using the staggered herringbone mixer at a flow rate of 12 mL/min; P=coefficient.

FIGS. 6A-6D are dot plots showing the number, rate, and reproducibility of DNA copies captured over time with 10 ml of plasma with 400 spiked-in BRAF^{Mut} DNA copies. Number of BRAF^{Mut} copies (FIG. 6A) and off-target ACTB copies (FIG. 6B) captured using the vortex-generating flow cell with the flow channel width of 0.5 mm and capture chamber width of 18 mm at a flow rate of 78 mL/min. Number of BRAF^{Mut} (FIG. 6C) copies and off-target ACTB copies (FIG. 6D) captured using the staggered herringbone mixer at a flow rate of 12 mL/min; P=coefficient.

FIG. 7 is an illustration of the two units of the 3D printed magnetic stand. Ten magnets were placed into each of the holes (circles) in the magnetic stand.

FIGS. 8A and 8B are dot plots showing the number of captured BRAF^{Mut} (FIG. 8A) and ACTB DNA (FIG. 8B) copies using 10 mL of plasma with 4,000 spiked in BRAF^{Mut} copies in the vortex-generating flow cell with a 0.5 mm wide flow channel and 18 mm wide capture chamber at a flow rate of 78 mL/min for 20 min using 1, 2, 4 or 8-fold the number of dCas9 capture molecules; P=coefficient.

FIGS. 9A-9D show exemplary devices comprising a channel 200, a sample inlet 300, a sample outlet 400, and chamber mixing elements 500 in FIGS. 9A-9C and microstructure mixing elements 510 in FIG. 9D.

### DETAILED DESCRIPTION

The present disclosure provides devices for the selective capture of ctDNA in plasma from a subject without alteration of the blood plasma, thus allowing the plasma to be returned to the subject. The disclosed methods allow detectable levels of ctDNA to be captured from plasma for cancer detection. Due to the selectivity for ctDNA, plasma samples can be aliquoted for other cancer detection methods (e.g., proteomics, methylation markers, and the like) following ctDNA separation.

Catalytically dead Cas9 (dCas9) and a guide RNA was used to capture BRAF^{Mut} DNA copies from a sample. Four passive microfluidic mixer flow cells were constructed to investigate the effects of microfluidic design on the capture rate of spiked-in BRAF T1799A (BRAF^{Mut}) ctDNA in unaltered flowing plasma. Once the capture rate was identified, the effects of the design of the microfluidic device, flow rate, flow time, and number of spiked-in mutant DNA copies were analyzed. Size modifications to the flow channel had no effect on the capture rate of ciDNA.

However, decreasing the capture chamber size decreased the flow rate required to achieve the capture rate. At the capture rate, the microfluidic design and flow rate did not affect the rate or number of captured ctDNA copies.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

“Biological sample,” as used herein, refers to any suitable sample obtained from any subject, as described here, and includes biological fluids, including, but not limited to, whole blood, serum, plasma, synovial fluid, cerebrospinal fluid, bronchial lavage, ascites fluid, bone marrow aspirate, pleural effusion, urine, as well as tumor tissue or any other bodily constituent or any tissue culture supernatant.

A “biomarker” includes a biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, a cell fragment, an exosome, and a small molecule, that is present in a biological sample and that may be isolated from, or measured in, the biological sample. Furthermore, a biomarker may be the entire intact molecule, or a portion thereof that may be partially functional or recognized, for example, by an antibody or other specific binding protein. A biomarker may be associated with a given state of a subject, such as a particular stage of disease. In some embodiments, the biomarker is a cancer biomarker (e.g., circulating tumor DNA, protein biomarkers (e.g., prostate specific antigen, alpha-fetoprotein, carcinoembryonic antigen). A measurable aspect of a biomarker may include, for example, the presence, absence, or concentration of the biomarker in the biological sample from the subject and/or relative changes of any of the measurable aspects compared to a standard (e.g., internal or from a healthy subject). The measurable aspect may also be a ratio of two or more measurable aspects of two or more biomarkers. Biomarker, as used herein, also encompasses a biomarker profile comprising measurable aspects of two or more individual biomarkers. The two or more individual biomarkers may be from the same or different classes of biomarkers such as, for example, a nucleic acid and a carbohydrate, or may measure the same or different measurable aspect such as, for example, absence of one biomarker and concentration of another. A biomarker profile may comprise any number of individual biomarkers or features thereof. In another embodiment, the biomarker profile comprises at least one measurable aspect of at least one internal standard. Methods of identifying and quantifying biomarkers are well known in the art and include histological and molecular methods such as enzyme-linked immunosorbent assays (ELISA) and other immunoassays, gel electrophoresis protein and DNA arrays, mass spectrometry, colorimetric assays, electrochemical assays, and fluorescence methods.

The term “biomolecule(s)” as used herein refers to molecules typically produced by living organisms. These molecules may include peptides, proteins, glycoproteins, nucleic acids, fatty acids or lipids, and sugars, that exist extracellularly or intracellularly.

The term “contacting” as used herein refers to bring or put in contact, to be in or come into contact. The term “contact” as used herein refers to a state or condition of touching or of immediate or local proximity.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice, and guinea pigs, and the like. Examples of non-mammals include, but are not limited to, birds, fish, and the like. In one embodiment, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Devices and Systems

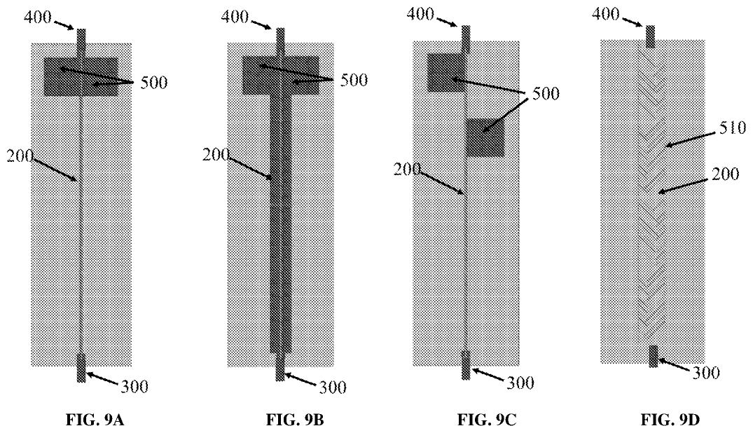

Disclosed herein are devices and systems suitable for capturing DNA from a sample. As shown in FIGS. 9A-9D, the devices comprise: a sample inlet 300; a sample outlet 400; a channel 200 fluidically connecting the sample inlet and sample outlet, wherein channel 200 comprises at least one mixing element (500 (FIGS. 9A-9C) and 510 (FIG. 9D)); and a plurality of capture complexes each complex comprising an RNA-guided DNA binding protein, or a functional fragment thereof, bound to a guide RNA configured to at least partially hybridize to a DNA of interest.

In some embodiments, the device is a microfluidic device. The term “microfluidic” as used herein is to be understood, without any restriction thereto, to refer to structures or devices through which fluid(s) are capable of being passed or directed, wherein one or more of the dimensions of any one or more microchannels is less than 1 millimeter. In some embodiments, the device is a macrofluidic device. The term “macrofluidic” as used herein is to be understood, without any restriction thereto, to refer to structures or devices through which fluid(s) are capable of being passed or directed, wherein the dimensions of any one or more macrochannels is greater than 1 millimeter.

The systems comprise a plurality of capture complexes each complex comprising an RNA-guided DNA binding protein, or a functional fragment thereof, bound to a guide RNA configured to at least partially hybridize to a DNA of interest; and a microfluidic device comprising: a sample inlet 300; a sample outlet 400; and a channel 200 fluidically connecting the sample inlet and sample outlet, wherein the channel comprises at least one mixing element (500 and 510).

In some embodiments, the DNA of interest is circulating free DNA (cfDNA), such that the capture complexes comprise a guide RNA configured to at least partially hybridize to a cfDNA of interest.

### a) Channel and Mixing Elements

A device according to the present invention comprises at least one channel 200 fluidically connecting a sample inlet 300 and a sample outlet 400. The term “channel” is not intended to be restricted to elongated configurations where the transverse or longitudinal dimension greatly exceeds the diameter or cross-sectional dimension. Rather, the term is meant to include a conduit of any desired shape or configuration through which liquids may be directed.

Channel 200 is defined by internal surfaces, which can also be referred to as channel surfaces or walls. The channel defines a fluid flow path between the inlet and outlet corresponding to the length of the channel. The channel has a width and depth which may vary or be constant along the length of the channel.

Channel 200 may be any configuration, having curves, spirals, turns, and the like. In some embodiments, the channel is substantially linear. A substantially linear channel is one in which no more than about 10% of the length of the channel deviates from a straight line extending between the inlet and outlet.

In some embodiments, channel 200 comprises a mixing element (500 and 510) to achieve mixing of the constituents a fluid sample along the length of the channel. The mixing element may be a passive mixing element. For example, the mixing element may be based on the structure of the channel or structures within the channel which enhance molecular diffusion and chaotic advection to all desired levels of mixing. Any type of passive mixing element may be employed in the disclosed devices including two-dimensional passive mixers with simple planar structures such as obstacles, unbalanced collisions, convergence-divergence channels, and spiral channels and three-dimensional passive mixers such as lamination based mixers and chamber based mixers.

In some embodiments, the mixing element comprises a plurality of microstructures or obstacles (510 in FIG. 9D). For example, the plurality of mixing microstructures may include ridges, channels, protrusions, or any combination thereof. The microstructures (510) may be located on any of the channel surfaces. In some embodiments, the microstructures span the entire length of the channel. In some embodiments, the microstructures cover on a portion of the total length of the channel. In some embodiments, the channel comprises two or more different types or arrangements of microstructures which are each located along select portions of the channel. The number and type of microstructures may be configured to create a specific level of mixing over a given length of the channel.

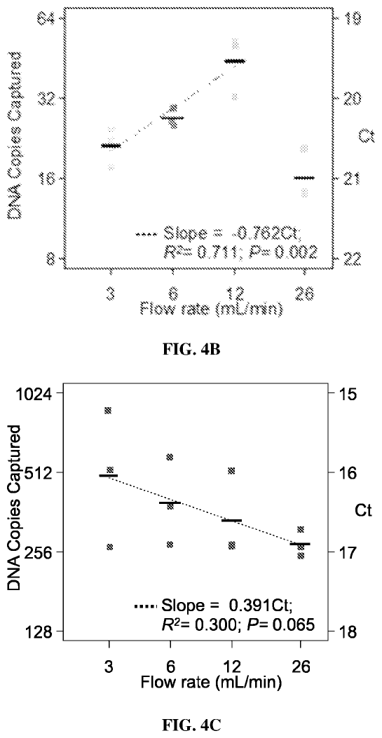

In select embodiments, the microstructures may create an alternating herringbone pattern, as shown in FIGS. 4A and 9D. The design consists of a herringbone pattern of ridges wherein the center of the herringbone pattern shifts from one side of the flow channel to the other and back again along the length of the flow channel. The alternating herringbone pattern likely causes the fluid in the channel to enter the channel and begin rotating in a first direction. Then, when the flowing fluid encounters a shifted herringbone pattern, the fluid is forced to rotate in a direction opposite to the first rotational direction.

The microstructures are not limited by height or depth. The height or depth of the plurality of the microstructures may be between 10-90% of the total height of the flow channel. In some embodiments, the height or depth of the microstructures may be about 20%, about 30%, about 40%, about 50%, about 60%, about 70%, about 80% or about 90% of the total height of the flow channel. In some embodiments, the height or depth of the microstructures may be between 20% and 80%, between 30% and 80%, between 40% and 80%, between 50% and 80%, between 60% and 80%, between 70% and 80%, between 20% and 70%, between 30% and 70%, between 40% and 70%, between 50% and 70%, between 60% and 70%, between 20% and 60%, between 30% and 60%, between 40% and 60%, between 50% and 60%, between 20% and 50%, between 30% and 50%, between 40% and 50%, between 20% and 40%, between 30% and 40%, or between 20% and 30% of the total height of the flow channel.

In some embodiments, the height or depth of the microstructures may vary along the length of the channel, may vary between a single portion of microstructures, or may vary between different types or arrangements of microstructures.

Alternatively, or in addition, in some embodiments, the mixing element comprises one or more chambers (500 in FIGS. 9A-9C). The one or more chambers (500) are configured to generate a vortex in the chamber based on the fluid dynamic principal that as a fluid travels through a narrow channel and then expands into a larger chamber vortices of fluid flow are formed in the chamber. Thus, the one or more chambers (500) span the width of channel 200 and expand out in at least one direction from the direction of sample flow. As a result, both the vortex in chamber 500 and the flow in channel 200 provide adequate mixing of the fluid.

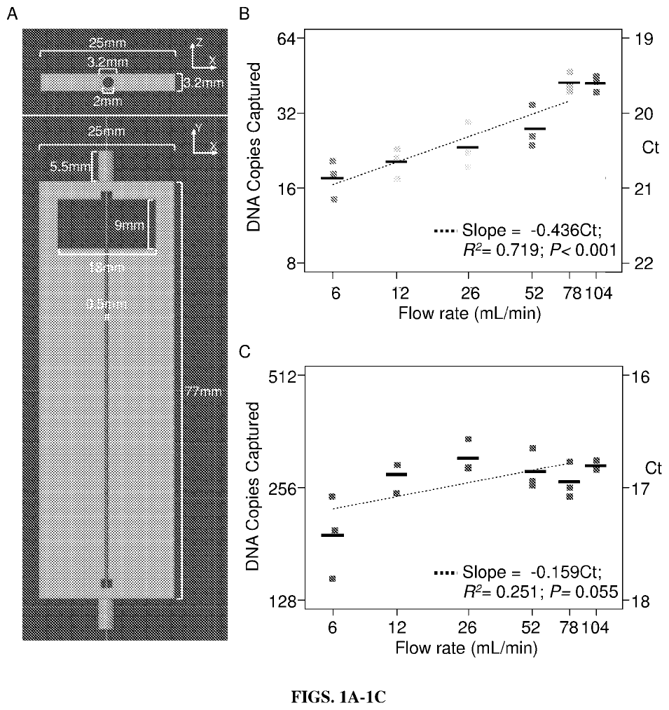

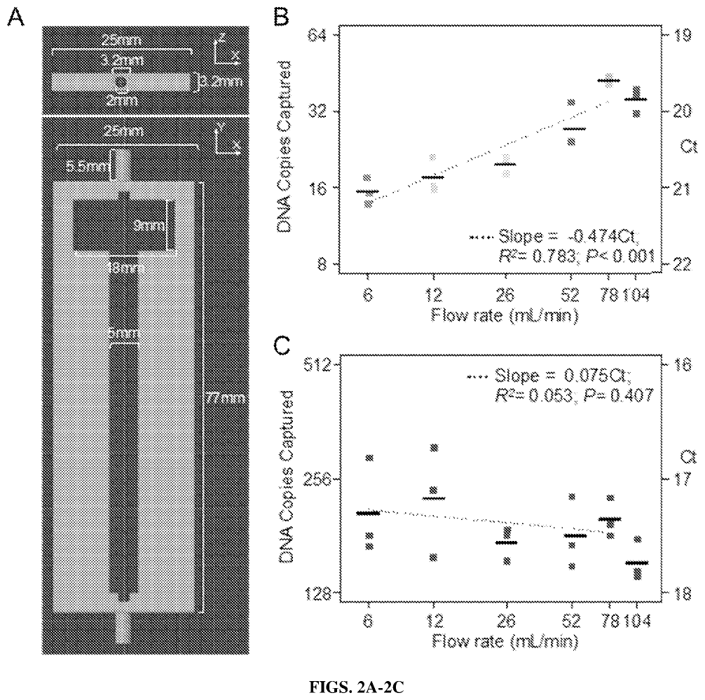

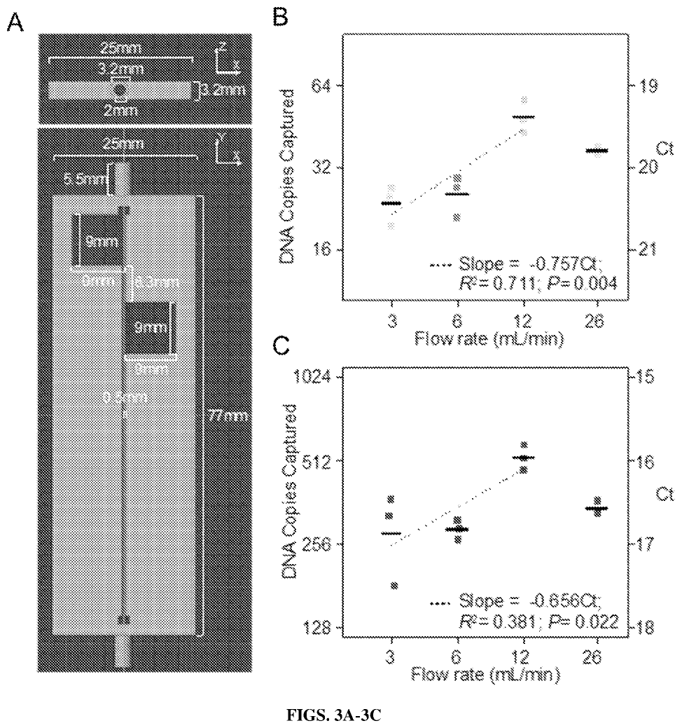

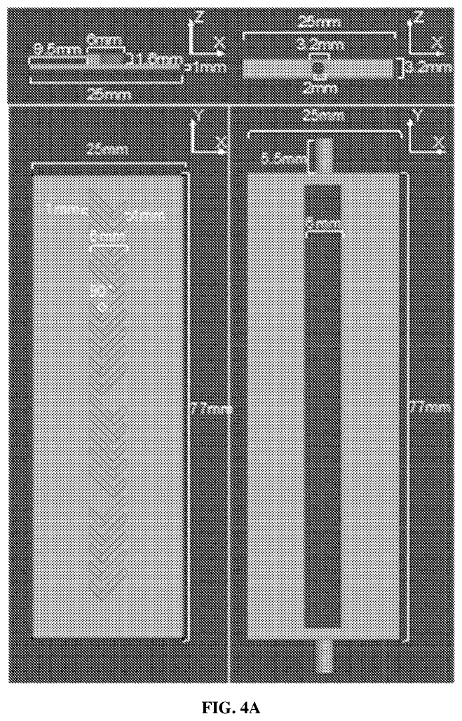

In some embodiments, the device may comprise a single chamber along the length of the channel. In some embodiments, the device may comprise multiple chambers along the length of the channel. The multiple chambers may be perpendicular to fluid flow in the same or different direction. The multiple chambers may be perpendicular to fluid flow in the same or different plane. For example, if fluid is flowing along a channel in an x axis, the chambers may expand away from the x-axis in the direction of the y axis or z axis. In some embodiments, each chamber may be perpendicular to fluid flow in the same plane but opposite directions. In some embodiments, each chamber may be perpendicular to fluid flow in the same plane and direction. The multiple chambers may be separated by a portion of the channel not encompassed in a chamber (see, for example, FIGS. 3A and 9C) or may span at least a portion of the same length of the channel, creating a single larger chamber which expands out in multiple directions from the fluid flow (see, for example, FIGS. 1A, 2A and 9A-9B).

The chambers may adopt any size or shape which allow expansion from the narrow channel into a larger chamber. For example, the chambers may be symmetric or asymmetric, relative to the channel. The chambers may be square, rectangle, convex, semi-circle, triangle, and/or fishbone shaped. In some embodiments, the height or depth of the chambers may be the same as the channel height. In some embodiments, the height or depth of the chambers may be different from the channel height. In some embodiments, the height or depth of two or more chambers is the same. In some embodiments, the height or depth of two or more chambers is the different.

The device is not limited by the material or substrate used for fabrication of any of the components (e.g., the channels or mixing elements). In some embodiments, the device is fabricated from a materials selected from the group consisting of silicon, fused-silica, glass, a polymer, a metal, an elastomer, polydimethylsiloxane, agarose, and a hydrogel, or any combination thereof. For example, in some embodiments, the channels and/or mixing elements are fabricated from a material selected from the group consisting of silicon, fused-silica, glass, polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), or any combination of these materials. The channels and/or mixing elements may be coated or uncoated for increased flow and decreased non-specific interaction of components of the sample.

### b) Capture Complexes

The capture complexes comprise an RNA-guided DNA binding protein, or a functional fragment thereof, bound to a guide RNA configured to at least partially hybridize to a DNA of interest. A functional fragment of an RNA-guided DNA binding protein comprises a region or domain for binding and associating with the guide RNA and, optionally, a region or domain for DNA binding or stabilization of the RNA bound to a corresponding nucleic acid.

In some embodiments, the RNA-guided DNA binding protein is a CRISPR-associated (Cas) protein. The Cas molecule can be from any Type or Class of CRISPR-Cas systems (e.g., Class 1, Class 3, Types I-VI, or any of subtypes thereof). Exemplary Cas proteins include: Cas1, Cas2, Cas3, Cas4, Cas5, Cash, Cas7, Cas8, Cas9, Cas10, Cas12, Cas13, and the like.

In some embodiments, the RNA-guided DNA binding protein is Cas9, or a fragment thereof. The Cas9 protein can be obtained from any suitable microorganism, and a number of bacteria express Cas9 protein orthologs or variants. Cas9 proteins of other species are known in the art (see, e.g., U.S. Patent Application Publication 2017/0051312, incorporated herein by reference) and may be used in connection with the present disclosure. The amino acid sequences of Cas proteins from a variety of species are publicly available through the GenBank and UniProt databases. The Cas 9 protein may be from Streptococcus pyogenes, Staphylococcus aureus (S. aureus), Campylobacter jejuni, Corynebacterium diphtheria, Eubacterium ventriosum, Streptococcus pasteurianus, Lactobacillus farciminis, Sphaerochaeta globus, Azospirillum (strain B510), Gluconacetobacter diazotrophicus, Neisseria cinerea, Roseburia intestinalis, Parvibaculum lavamentivorans, Nitratifractor salsuginis (strain DSM 16511), Campylobacter lari (strain CF89-12), or Streptococcus thermophilus (strain LMD-9). In some embodiments, the Cas9 is from Streptococcus pyogenes or Staphylococcus aureus.

In some embodiments, the Cas9 protein is a Cas9 nickase (Cas9n). Wild-type Cas9 has two catalytic nuclease domains facilitating double-stranded DNA breaks. A Cas9 nickase protein is typically engineered through inactivating point mutation(s) in one of the catalytic nuclease domains causing Cas9 to nick or enzymatically break only one of the two DNA strands using the remaining active nuclease domain. Cas9 nickases are known in the art (see, e.g., U.S. Patent Application Publication 2017/0051312, incorporated herein by reference) and include, for example, Streptococcus pyogenes with point mutations at D10 or H840. In select embodiments, the Cas9 nickase is Streptococcus pyogenes Cas9n (D10A).

In some embodiments, the Cas9 protein is a catalytically-dead Cas9. Catalytically-dead dCas9 can be obtained, for example, by introducing point mutations (e.g., substitutions, deletions, or additions) in the Cas9 molecule at the DNA-cleavage domain, e.g., the nuclease domain, the RuvC and/or HNH domain. See, e.g., Jinek et al., Science (2012) 337:816-21, incorporated by reference herein in its entirety. For example, introducing two point mutations in the RuvC and HNH domains reduces the Cas9 nuclease activity while retaining the Cas9 RNA and DNA binding activity. For example, Streptococcus pyogenes Cas9 may be rendered catalytically dead by mutations of D10 and at least one of E762, H840, N854, N863, or D986, typically H840 and/or N863A (see, e.g., U.S.

Patent Application Publication 2017/0051312, incorporated herein by reference). Mutations in corresponding orthologs are known, such as N580 in Staphylococcus aureus Cas9. Similar mutations can also apply to any other naturally-occurring Cas9 (e.g., Cas9 from other species) or engineered Cas9 molecules. Oftentimes, such mutations cause catalytically-dead Cas9 to possess no more than 3% of the normal nuclease activity.

The gRNA may be a crRNA, crRNA/tracrRNA (or single guide RNA, sgRNA). The terms “gRNA,” “guide RNA,” and “CRISPR guide sequence” may be used interchangeably throughout and refer to a nucleic acid comprising a sequence that determines the binding specificity of the RNA-guided DNA binding protein. A gRNA hybridizes to (complementary to, partially or completely) the target cfDNA.

The gRNA or portion thereof that hybridizes to the cfDNA may be between 15-40 nucleotides in length. In some embodiments, the gRNA sequence that hybridizes to the cfDNA is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLoS ONE, 10(3): (2015)); Zhu et al. (PLoS ONE, 9(9) (2014)); Xiao et al. (Bioinformatics. Jan. 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer.

There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to the DNA of interest, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096): 816-821, and Ran, et al. Nature Protocols (2013) 8:2281-2308, incorporated herein by reference in their entireties.

In some embodiments, the plurality of capture complexes comprises more than one type of capture complex each having a guide RNA configured to at least partially hybridize to a different DNA of interest. In some embodiments, the plurality of capture complexes comprises more than one type of capture complex each having a different guide RNA configured to at least partially hybridize to a single DNA of interest. For example, when the DNA of interest is cfDNA, the plurality of capture complexes may comprise more than one type of capture complex each having a different guide RNA configured to at least partially hybridize to a single cfDNA of interest or may comprise more than one type of capture complex each having a guide RNA configured to at least partially hybridize to a different cfDNA of interest.

In some embodiments, the plurality of capture complexes are immobilized on one or more interior surfaces of the channel. In some embodiments, the plurality of capture complexes are immobilized on one or more interior surfaces of the one or more chambers or the mixing element microstructures.

The immobilized capture complexes are stationary compared to the direction of flow. Any method of immobilizing the capture complexes, may be utilized with devices and systems of the present invention. For example, the capture complexes may be immobilized by a covalent linkage to a surface or substrate, the capture complexes may be immobilized magnetically or electrochemically, the capture complexes may be immobilized through immunoprecipitation (e.g., with anti-Cas9, biotin, or epitope tag (e.g., FLAG tag) antibody or binding partner conjugated beads), or the capture complexes may be immobilized physically by partitioning into distinct areas of the channel. Capture complexes may be immobilized on or within any portion of the channel.

In some embodiments, the plurality of capture complexes are magnetically immobilized to the one or more locations on one or more interior surfaces of the one or more chambers or the mixing element microstructures.

Thus, in some embodiments, the devices and systems further comprise at least one magnet. The at least one magnet may be configured to be arranged above, below, or around at least a portion of the device. The magnet may be configured to be actuated, such that the capture complexes are immobilized on or within a surface of the flow cell or within the fluidic chamber when magnet is engaged.

In some embodiments, the capture complexes are linked to magnetic particles. “Magnetic particle,” as used herein, refers to so-called magnetic beads, magnetic microbeads, paramagnetic particles, magnetically attractable particles, magnetic spheres, and magnetically responsive particles. These terms are often used interchangeably throughout the field. As such, “magnetic particle” includes any of the particles capable of being manipulated in a liquid with the application of a magnetic field. The magnetism of the bead may include paramagnetic, superparamagnetic, ferromagnetic, antiferromagnetic, and ferrimagnetic properties.

In some embodiments, the capture complex is linked to the magnetic particle by a biotin streptavidin linker. For example, the RNA-guided DNA binding protein may comprise a biotin tag and the particle comprises streptavidin or similar biotin-binding agent.

The RNA-guided DNA binding protein may comprise an epitope tag (e.g., a FLAG tag, an HA tag, a Myc tag, and the like). In some embodiments, the RNA-guided DNA binding protein comprises a FLAG tag. The biotin and/or epitope tags may be located at the N-terminus, a C-terminus, or a combination thereof.

The devices may be connected to a fluidic system configured to introduce a sample into the device, then remove the sample from the device, and, optionally, re-introduce the sample to device. In some embodiments, the fluidic system is further configured to deliver buffer or wash solution to the device. The buffer or wash solution may utilize the same or different inlet or outlet to the device. The fluidic system may include, for example, pumps and valves that are selectively operable for controlling fluid communication within a single device or between multiple devices.

The disclosed devices may allow for multiplexing, e.g., allow for loading of multiple devices in the sample path. Furthermore, the devices and systems disclosed herein can be integrated with other devices to allow multistep processes. For example, the devices or system can be integrated with other analysis devices for detection of other biomarkers and/or sequencing of the DNA of interest.

The systems may further comprise reagents for amplifying and/or sequencing the DNA of interest. Many such reagents are known in the art and commercially available. Examples of suitable reagents include conventional reagents employed in nucleic acid amplification reactions, such as, for example, one or more enzymes having polymerase activity, enzyme cofactors (such as magnesium or nicotinamide adenine dinucleotide (NAD)), salts, buffers, deoxyribonucleotide, or ribonucleotide triphosphates (dNTPs/rNTPs; for example, deoxyadenosine triphosphate, deoxyguanosine triphosphate, deoxycytidine triphosphate, and deoxythymidine triphosphate) blocking agents, labeling agents, and the like. Other reagents used in amplification reactions include nicking enzymes, single-strand binding proteins, helicases, resolvases, and the like.

One or more of the components of the system may be provided as a composition. The compositions may further comprise carriers. Carriers may include any and all solvents, dispersion media, antibacterial and antifungal agents, isotonic and absorption delaying agents. Some examples of materials which can serve as carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, preservatives, and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

The compositions may also comprise buffers (e.g., neutral buffered saline or phosphate buffered saline), carbohydrates (e.g., glucose, mannose, sucrose or dextrans), mannitol, antioxidants, bacteriostats, chelating agents such as EDTA or glutathione, solutes that render the formulation isotonic, hypotonic, or weakly hypertonic with the blood of a recipient, suspending agents, thickening agents and/or preservatives, commonly found in proteinaceous compositions.

As used herein, the term “sample” is used in its broadest sense. In one sense, it is meant to include a specimen obtained from any source, including biological, environmental, and laboratory samples. Biological samples may be obtained from animals (including humans) and encompass fluids, solids, and/or tissues. Such examples are not however to be construed as limiting the sample types. In some embodiments, the sample is a fluid sample such as a liquid sample. Examples of liquid samples suitable for use with the devices disclosed herein include bodily fluids (e.g., blood, serum, plasma, saliva, urine, ocular fluid, semen, sputum, sweat, tears, and spinal fluid), water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. Liquid samples can be made from solid, semisolid, or highly viscous materials, such as fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample. Residual particulates may be removed or reduced using conventional methods, such as filtration or centrifugation. Samples can comprise biological materials, such as cells, microbes, organelles, and biochemical complexes.

In certain embodiments, the systems further comprise a biological sample. The biological sample can be any suitable sample obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as, a nasal swab or brush, or a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, and the like. Samples may also be obtained from live or dead organisms or from in vitro cultures. In some embodiment, the biological sample is a fluid, such as human blood (serum or plasma), synovial fluid, cerebrospinal fluid, urine, prostatic fluid, lymph fluid, saliva, and tracheal lavage fluid. In some embodiments, the biological sample comprises blood or blood components. In some embodiments, the biological sample comprises plasma.

In some embodiments, the sample is a biological sample obtained from a subject having or suspected of having a disease or disorder.

In some embodiments, the devices and systems comprise a computer (or processor) and computer-readable media for providing a user interface as well as manual, semi-automated, or fully-automated control of all functions, e.g., control of the fluidics system (e.g., volumetric fluid flow rates, fluid flow velocities, the timing and duration for sample and bead addition, reagent addition, and rinse steps), the temperature control system (e.g., specifying temperature set point(s)), and integration with other equipment or devices. In some embodiments, the computer or processor may be an integrated component of the device. In some embodiments, the computer or processor may be a stand-alone module, for example, a personal computer or laptop computer.

### 3. Methods of Capturing Dna

The present disclosure provides methods of capturing DNA from a sample. The methods comprise introducing the sample into a device as disclosed herein to form a complex comprising a capture complex and the DNA of interest. In some embodiments, the sample is a biological sample. In some embodiments, the DNA of interest is circulating free DNA (cfDNA).

Descriptions of the capture complexes set forth above in connection with the inventive devices and systems also are applicable to the disclosed methods.

The sample may be flowed through a device at any flow rate or for any amount of time.

One of skill in the art can adjust the flow rate and time based on the binding kinetics of the interaction between the capture complex and the DNA of interest. The flow rate is desirably maintained for a sufficient period of time to allow for the binding interaction between the capture complex and the DNA to occur.

The sample may be introduced, for example, at a flow rate of about 3 mL/min to about 120 mL/min. In some embodiments, the flow rate is about 3 mL/min, about 5 mL/min, about 10 mL/min, about 20 mL/min, about 30 mL/min, about 40 mL/min, about 50 mL/min, about 60 mL/min, about 70 mL/min, about 80 mL/min, about 90 mL/min, about 100 mL/min, about 110 mL/min, or about 120 mL/min. In some embodiments, the same is introduced at a flow rate of 3-110 mL/min, 3-100 mL/min, 3-90 mL/min, 3-80 mL/min, 3-70 mL/min, 3-60 mL/min, 3-50 mL/min, 3-40 mL/min, 3-30 mL/min, 3-20 mL/min, 3-10 mL/min, 5-110 mL/min, 5-100 mL/min, 5-90 mL/min, 5-80 mL/min, 5-70 mL/min, 5-60 mL/min, 5-50 mL/min, 5-40 mL/min, 5-30 mL/min, 5-20 mL/min, 5-10 mL/min, 10-110 mL/min, 10-100 mL/min, 10-90 mL/min, 10-80 mL/min, 10-70 mL/min, 10-60 mL/min, 10-50 mL/min, 10-40 mL/min, 10-30 mL/min, 10-20 mL/min, 20-120 mL/min, 20-110 mL/min, 20-100 mL/min, 20-90 mL/min, 20-80 mL/min, 20-70 mL/min, 20-60 mL/min, 20-50 mL/min, 20-40 mL/min, 20-30 mL/min, 30-120 mL/min, 30-110 mL/min, 30-100 mL/min, 30-90 mL/min, 30-80 mL/min, 30-70 mL/min, 30-60 mL/min, 30-50 mL/min, 30-40 mL/min, 40-120 mL/min, 40-110 mL/min, 40-100 mL/min, 40-90 mL/min, 40-80 mL/min, 40-70 mL/min, 40-60 mL/min, 40-50 mL/min, 50-120 mL/min, 50-110 mL/min, 50-100 mL/min, 50-90 mL/min, 50-80 mL/min, 50-70 mL/min, 50-60 mL/min, 60-120 mL/min, 60-110 mL/min, 60-100 mL/min, 60-90 mL/min, 60-80 mL/min, 60-70 mL/min, 70-120 mL/min, 70-110 mL/min, 70-100 mL/min, 70-90 mL/min, 70-80 mL/min, 80-120 mL/min, 80-110 mL/min, 80-100 mL/min, 80-90 mL/min, 90-120 mL/min, 90-110 mL/min, 90-100 mL/min, 100-120 mL/min, 100-110 mL/min, or 110-120 mL/min.

In select embodiments, the mixing element comprises two chambers along a single portion of the channel, perpendicular to sample flow direction in the channel, and the flow rate is about 70 mL/min to about 120 mL/min. In certain embodiments, the mixing element comprises two chambers along a single portion of the channel, perpendicular to sample flow direction in the channel, and the flow rate is about 70 mL/min, about 80 mL/min, about 90 mL/min, about 100 mL/min, about 110 mL/min, or about 120 mL/min.

In select embodiments, the mixing element comprises two chambers along different portions of the channel, perpendicular to sample flow direction in the channel, and the flow rate is 10-30 mL/min. In certain embodiments, the mixing element comprises two chambers along different portions of the channel, perpendicular to sample flow direction in the channel, and the flow rate is about 10 mL/min, about 11 mL/min, about 12 mL/min, about 13 mL/min, about 14 mL/min, about 15 mL/min, about 16 mL/min, about 17 mL/min, about 18 mL/min, about 19 mL/min, about 20 mL/min, about 21 mL/min, about 22 mL/min, about 23 mL/min, about 24 mL/min, about 25 mL/min, about 26 mL/min, about 27 mL/min, about 28 mL/min, about 29 mL/min, or about 30 mL/min.

In select embodiments, the mixing element comprises a plurality of microstructures and the flow rate is 10-20 mL/min. In certain embodiments, the mixing element comprises a plurality of microstructures and the flow rate is about 10 mL/min, about 11 mL/min, about 12 mL/min, about 13 mL/min, about 14 mL/min, about 15 mL/min, about 16 mL/min, about 17 mL/min, about 18 mL/min, about 19 mL/min, or about 20 mL/min.

In some embodiments, the sample may be introduced to the device a single time. Alternatively, in some embodiments, the sample may be reprocessed over the device more than once to maximize exposure of the sample to the capture complexes.

The binding affinity between capture complex and the DNA of interest should be sufficient to remain bound under the conditions of the assay, including any wash steps used to remove molecules that are non-specifically bound. In some embodiments, the method comprises one or more wash steps.

As used herein, the term “sample” is used in its broadest sense. In one sense, it is meant to include a specimen obtained from any source, including biological, environmental, and laboratory samples. Biological samples may be obtained from animals (including humans) and encompass fluids, solids, and/or tissues. Such examples are not however to be construed as limiting the sample types. In some embodiments, the sample is a fluid sample such as a liquid sample. Examples of liquid samples suitable for use with the devices disclosed herein include bodily fluids (e.g., blood, serum, plasma, saliva, urine, ocular fluid, semen, sputum, sweat, tears, and spinal fluid), water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. Liquid samples can be made from solid, semisolid, or highly viscous materials, such as fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample. Residual particulates may be removed or reduced using conventional methods, such as filtration or centrifugation. Samples can comprise biological materials, such as cells, microbes, organelles, and biochemical complexes.

In some embodiments, the sample is a biological sample. The biological sample can be any suitable sample obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as, a nasal swab or brush, or a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, and the like. Samples may also be obtained from live or dead organisms or from in vitro cultures. In some embodiment, the biological sample is a fluid, such as human blood (serum or plasma), synovial fluid, cerebrospinal fluid, urine, prostatic fluid, lymph fluid, saliva, and tracheal lavage fluid. In some embodiments, the biological sample comprises blood or blood components. In some embodiments, the biological sample comprises plasma.

In some embodiments, the sample is a biological sample obtained from a subject having or suspected of having a disease or disorder.

The sample can be obtained from a subject using routine techniques known to those skilled in the art, and the sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like.

The total volume of the sample can vary depending on the type of sample and the molecule(s) of interest. In some embodiments, the sample have a volume less than 4,000 mL, less than 3,500 mL, less than 3,000 mL, less than 2,500 mL, less than 2,000 mL, less than 1,500 mL, less than 1,000 mL, less than 750 mL, less than 500 mL, less than 250 mL, less than 100 mL, less than 10 mL, less than 1 mL, less than 500 μL, less than 100 μL, less than 50 μL, less than 20 μL, or less than 10 μL.

The sample may be diluted prior to use in the systems and methods disclosed herein. The sample may be diluted about 1-fold, about 2-fold, about 3-fold, about 4-fold, about 5-fold, about 6-fold, about 10-fold, or greater, prior to use. The sample may be concentrated prior to use in the systems and methods disclosed herein. The sample may be concentrated about 1-fold, about 2-fold, about 3-fold, about 4-fold, about 5-fold, about 6-fold, about 10-fold, or greater, prior to use.

The devices, systems, and methods are not limited by the type or source of the DNA of interest. In some embodiments, the DNA of interest is circulating free DNA (cfDNA). Circulating free DNA (cfDNA) refers to extracellular DNA (single-stranded or double-stranded DNA) present in a biological sample. cfDNA may be derived from normal or diseased cells in the subject. The concentration, integrity, genetic, and epigenetic alternations in the cfDNA may suggest pathological conditions of the body, such as infection, inflammation, autoimmune diseases, and cancer. cfDNA comprises various forms of DNA freely circulating in body fluids, including, but not limited to, circulating tumor DNA (ctDNA), circulating cell-free mitochondrial DNA (ccf mtDNA), and cell-free fetal DNA (cffDNA). cfDNA can also be used to describe DNA derived from infectious agents. The present methods and systems are not limited by the type of cfDNA being detected.

In some embodiments, the circulating free DNA is cell free DNA (cfDNA) of microbial (e.g., bacterial, fungal, intracellular, or extracellular parasites) and viral (e.g., bacteriophages, eukaryotic viruses) origin.

In some embodiments, the circulating free DNA is circulating tumor DNA. Circulating tumor DNA (ctDNA) refers to DNA that comes from cancerous cells and tumors. The quantity of ctDNA varies among individuals and depends on the type of tumor, its location, and for cancerous tumors, the cancer stage. ctDNA may be specific for the type of cancer, stage of cancer, or location of cancer, although some ctDNA is found in a variety of different types of cancer. Oftentimes, ctDNA has mutations characteristic of the cancer or tumor cells from which it is derived.

In some embodiments, the methods further comprise removing remaining sample from the device. The removing may be completed simultaneous with the introducing, such that remaining sample is being removed from the device as a new sample is being introduced. Alternatively, the device may be charged with a certain volume of sample, such that the remaining sample is being removed from the device as a wash buffer, for example, is being introduced.

The methods disclosed herein may further comprise one or more of: purifying the DNA of interest; amplifying the DNA of interest; and sequencing the DNA of interest.

Purifying the DNA of interest may comprise separating the DNA from the capture complexes (e.g., changing the binding conditions or adding additional competitive binding agent to cause dissociation of DNA from capture complexes), digesting the protein components of the capture complexes, cleaning and concentrating the DNA, and the like.

In some embodiments of the disclosed methods, one or more nucleic acid amplification reactions may be performed to create multiple copies of the DNA of interest. Any suitable amplification method known in the art allowing for sensitive detection of DNA may be used, including by not limited to polymerase chain reaction (PCR), preferably real time PCR, especially probe-based methods such as Taq-Man, Scorpions, Molecular Beacons; and/or isothermal amplification. In some embodiments, the amplification utilizes probes or intercalating dyes for quantitative amplification. In some embodiments, the amplification utilizes barcodes, adapters, or other means for identifying the isolated DNA or generating primer binding cites for the DNA.

Following or concomitantly with amplification, the DNA or amplified nucleic acid may be sequenced. Sequencing can be accomplished using high-throughput systems, some of which allow detection of a sequenced nucleotide immediately after or upon its incorporation into a growing strand, e.g., detection of sequence in real time or substantially real time. In some embodiments, sequencing is achieved by next-generation sequencing. In some embodiments, the next-generation sequencing is chosen from the group consisting of pyrosequencing, single-molecule real-time sequencing, sequencing by synthesis, sequencing by ligation (SOLID sequencing) or nanopore sequencing.

In certain embodiments, the method further comprises analyzing at least a portion of the sample for the presence or absence of at least one additional biomolecule or agent. The analysis may be done before or after the capture of the DNA.

In some embodiments, the at least one additional agent is a biomarker. The biomarker may be any substance in which its presence, absence, or relative quantity in a subject may indicate a particular disease or stage of disease. Biomarkers have been linked to a number of diseases such as, cancer, diabetes, multiple sclerosis, neurodegenerative disorders, stroke, etc. Examples of commonly measured biomarkers in humans include proteins (e.g., cytokines, metabolic enzymes, cell cycle enzymes, cytoskeletal protein, autoantibodies, growth factors, and neuropeptides), hormones (e.g., steroid hormones, dehydroepiandrosterone (DHEA), estrogen, vasopressin, cholesterol, adrenalin, cortisol, and cortisone), metabolites (e.g., alcohol, lactic acid, lactate, urea, and creatinine), and small molecules (e.g., vitamins, glucose, penicillin, and hydrogen peroxide).

In some embodiments, the methods further comprise returning at least a portion of the biological sample to the subject. For example, once the DNA of interest is isolated from the plasma, the remaining plasma can be reintroduced back to the subject using known methods.

### 4. Kits

Also provided herein are kits comprising a device or system, or the components thereof, as described herein.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. The kits optionally may provide additional components such as buffers and disposable single-use equipment (e.g., pipettes, cell culture plates, flasks).

The kit can further contain control samples to be analyzed. Optionally, the kit includes quality control components (for example, sensitivity panels, calibrators, and positive controls).

If desired, the kit can further comprise one or more components, alone or in further combination with instructions, for assaying the test sample for another analyte, which can be a biomarker

The kits provided herein are in suitable packaging. Suitable packaging includes, but is not limited to, vials, bottles, jars, flexible packaging, and the like. Individual member components of the kits may be physically packaged together or separately.

### 5. Examples

### Materials and Methods

Microfluidic flow cell and magnetic stand construction The 3D modeling software Blender (Version 2.93.5) was used to design the microfluidic flow cells and magnetic stand. A 3D printer (Stratasys, Objet 260) was used to generate the flow cells and magnetic stand. Twenty magnets (K&J magnet, D42-N52) were inserted into the magnetic stand. Glass microscope slides (Fisher Scientific, 12-544-1) and/or the 3D printed herringbone insert were adhered to the flow cells with 100% silica adhesive (PPG, LN-207).

Flow cell design The width and length of the flow cells were chosen to have similar proportions as a common glass microscope slide (75 mm×25 mm). Glass microscope slides (Fisher Scientific, 12-544-1) and/or the 3D printed herringbone insert were adhered to the flow cells with 100% silica adhesive (PPG, LN-207). The size of the capture chamber of the vortex-generating flow cells was chosen to be the largest size possible while maintaining the surface area required for microscope slide adhesion. The length of the capture chamber was designed to be equal to the width of the capture chamber. The height of the flow cells was chosen to be the minimum height for an inlet that could fit 1.6 mm silicone tubing (Bio-Rad; 7318211). The minimum width of the flow channel was chosen because it was the lowest tolerance of the 3D printer. The channel width of the herringbone flow cell was selected to fit the magnetic stand. The height, width and spacing of the herringbone patterns were modeled to have similar proportions as a previously published herringbone flow cell.

Plasma and spiked-in BRAFMut DNA The plasma used in this study was pooled plasma from gender unspecific healthy individuals purchased from BioIVT (K₂EDTA plasma, BioIVT, Hicksville, NY). BRAF^{Mut} duplexed DNA modified with phosphorylated 3′ ends were purchased from Integrated DNA Technologies (IDT; Coralville, IA) and have been previously reported. The BRAF^{Mut} DNA was diluted with TE buffer (IDT, 11-05-01-05) mixed with 5 ng/μL of carrier RNA (Qiagen, 142342312) and was added to the plasma as indicated. dCas9 system assembly To block the metallic streptavidin beads, for each reaction, 8 μL of beads (Thermo Fisher, Dynabeads-Streptavidin M280) was incubated in 200 μL of 1× rCutSmart buffer (NEB, B6004S) for 30 min at room temperature. A countersunk ring magnet (K&J magnet, RX033CS-P-N52) was used to pull down the metal beads. After 30 min, the blocked beads were washed 2 times with 100 μL of 1× rCutSmart buffer and resuspended with 10 μL of 1× rCutSmart buffer per reaction.

To assemble the ribonucleoproteins (RNPs), for each reaction, 5 pmol of dCas9-3×FLAG™ Biotin Protein (MilliporeSigma) was incubated with 50 pmol sgRNA (IDT) for 30 min at room temperature. To assemble the RNPs beads, the blocked beads were mixed with the assembled RNPs and incubated for 30 min at room temperature. The RNP-assembled bead mixture was then pulled down with a countersunk ring magnet and washed 3 times with 100 μL of 1× rCutSmart buffer and resuspended in 10 μL of 1× rCutSmart buffer per reaction.

dCas9 ctDNA capture in the flow cell devices A peristaltic pump was used to pump the plasma through the flow cell devices. A 15 mL polypropylene tube (Corning, 10086) was used as the plasma reservoir. After the capture was completed, the flow cell was removed from the magnetic stand. The dCas9 molecules were then resuspended within the flow cell and recaptured in a 0.5 mL microcentrifuge tube using a ring magnet (K&J magnet, R844-N52). After the plasma was removed, the beads were washed 3 times with filtered sterilized dCas9 washing buffer (50 mM Tris (pH 8.0), 150 mM NaCl, and 0.05% Tween 20). The washed RNP beads were resuspended in 45 μL TNES buffer (10 mM Tris, 150 mM NaCl, 2 mM EDTA, 05% SDS) and digested with 5 μL proteinase K (Invitrogen, 25530049) at 55° C. for 1 hour. After proteinase K digestion, a DNA clean & concentrator-5 kit (Zymo, D4013) was used to clean and concentrate the captured DNA. After the captured DNA was cleaned, each reaction was eluted with 6 μL of EB buffer (Zymo, D3004-4-50). The captured and concentrated DNA was amplified and measured using allele specific duplex PCR of which protocol, primers and probe sequences have been previously reported 14. Quantification of the captured and spiked-in DNA were calculated from measurements by nanodrop (NanoDrop 2000, Thermo Scientific) or real-time PCR calculations.

RStudio (Version 1.2.1335) using R (version 3.6.0) and custom code was used for all of the statistical analyses and the generation of the plots in this study.

### Example 1

### Devices and Capture Rate of ctDNA

To investigate how microfluidic device design and passive microfluidic mixing flow cells can affect the capture rate of ctDNA in unaltered flowing plasma, a 3D printer was used to generate four passive microfluidic mixer flow cells. The four printed flow cells were closed by adhering glass microscope slides and/or the herringbone insert onto the printed flow cells with 100% silica adhesive. A magnetic stand was printed to standardize the immobilization of the dCas9 capture molecules in the flow cells (FIG. 7). The magnetic stand is composed of two separate units that hold 10 magnets per unit and was designed to flank both sides of the flow cell. Four of the twenty magnets in the magnetic stand were used to immobilize the dCas9 capture system in each of the flow cell devices. To avoid an accumulation of air bubbles in the flow cell, the unaltered plasma was pumped upwards through the device while it was set in the vertical position.

The BRAF^{Mut} oncogene, also known as BRAF (p. V600E), was chosen as the target because it is one of the most frequently mutated oncogenes and can be targeted with BRAF inhibitor treatments. Since studies have shown that, in cancer patients, the mutant allele fraction (MAF) (calculated as the number of ctDNA copies/total number of circulating cfDNA copies) ranges from undetectable to >80%, 4,000 copies of BRAF^{Mut} DNA were spiked into 10 mL of unaltered human plasma from healthy individuals for the majority of the experiments in this study, as this copy number has previously been shown to be the equivalent of 10% MAF. Although the single guide RNA (sgRNA) drives capture specificity for the dCas9 protein, it is recognized that off-target capture can occur. To investigate the effects of off-target binding, both on-target BRAF^{Mut} DNA and off-target ACTB DNA capture were measured throughout this study.

The first three microfluidic devices were designed to generate vortexes by using the same fluid dynamic principle. As fluid travels, at an appropriate flow rate, through a narrow channel that expands into a larger chamber, vortices are formed within the chamber. The first vortex-generating flow cell was designed to have a small flow channel (0.5 mm width) and a large capture chamber (18 mm width) (FIG. 1A). For clarity, this flow cell has been named the basic vortex-generating flow cell. To investigate if the size of the flow channel, which changes the Reynolds number, affects the capture rate of ctDNA, the second vortex-generating flow cell was designed to have a 10-fold wider flow channel (5.0 mm width) in comparison to the basic vortex-generating flow cell (FIG. 2A). This flow cell was named the wide flow channel vortex-generating flow cell. To investigate if the size of the capture chamber affected the capture rate of ctDNA, the third vortex-generating flow cell was designed to have two half-sized (9 mm width) capture chambers as compared to the basic vortex-generating flow cell (FIG. 3A). This flow cell was named the small capture chambers vortex-generating flow cell. To investigate if passive mixing flow cells that have different fluid dynamic principle affects the capture rate of ctDNA, the fourth flow cell was designed to cause turbulent flow by using a staggered herringbone mixer (SHM). SHMs use repeated patterns of grooves to generate chaotic twisting flow and efficient as a passive microfluidic mixer. This flow cell was designed to have a positive pattern SHM and was used with forward flow which has been shown to have high mixing efficiency (FIG. 4A). This flow cell was named the SHM flow cell.

In the basic vortex-generating flow cell, after 4,000 copies of BRAF^{Mut} DNA were spiked into 10 mL of unaltered plasma, a peristaltic pump was used to flow the plasma, at a range of flow rates between 6 and 104 mL/min for 20 minutes, across the dCas9 capture system in the flow cell. After 20 minutes of flow, the number of BRAF^{Mut} DNA copies and off-target ACTB DNA copies captured by the dCas9 capture system were measured using allele specific qPCR. Between the flow rates of 6 and 78 mL/min, the number of captured BRAF^{Mut} copies increased as the flow rate increased with linearity (Slope=−0.436 Ct; R²=0.719; coefficient P<0.001) (FIG. 1B). The number of captured BRAF^{Mut} DNA copies peaked at 78 mL/min with, on average, 42 copies of BRAF^{Mut} DNA captured. Between the flow rates of 6 and 78 mL/min, no correlation between the number of captured off-target ACTB DNA copies and the flow rate was found (Slope=−0.159 Ct; R²=0.251; coefficient P=0.055) (FIG. 1C).

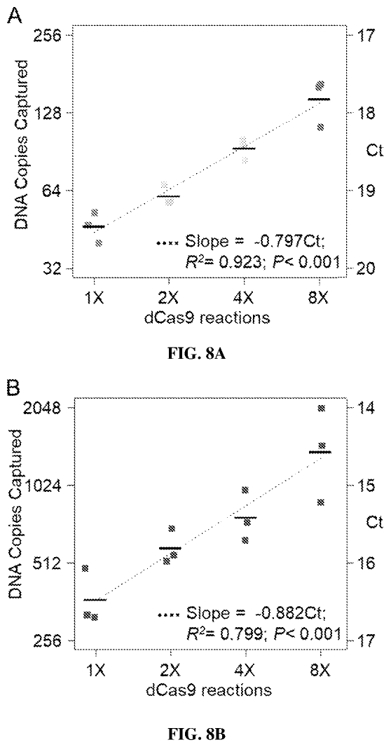

To investigate if increasing the number of dCas9 molecules to the flow cell would increase the number or rate of captured DNA, 1, 2, 4, and 8-fold more dCas9 molecules were added into the vortex-generating flow cell with the 0.5 mm flow channel and 18 mm capture chamber. After flowing 10 mL of plasma with 4,000 spiked-in BRAF^{Mut} DNA copies at a flow rate of 78 mL/min for 20 minutes, the number of BRAF^{Mut} DNA copies and off-target ACTB DNA copies captured increased as the number of dCas9 molecules increased for both BRAF^{Mut} and ACTB DNA (Slope=0.797 Ct; R²=0.923; coefficient P <0.001) and (Slope=−0.882 Ct; R²=0.799; coefficient P<0.001), respectively (FIGS. 8A and 8B).

Surprisingly, a saturation of dCas9 molecules was unable to be reached even though the accumulated surface area covered by the streptavidin beads, in a 1-fold reaction, is 249-fold more than the surface area of the four magnets used to immobilize the dCas9 molecules in the flow cells. However, since the diameter of DNA is 2 nm, over 1000 times smaller than the diameter of the 2.8 um streptavidin metal bead, it was possible that the ctDNA can permeate through the multilayered streptavidin beads within the flow cell. This result provided evidence that both the surface area and the strength of the magnet, which can affect the ability to layer the streptavidin metal beads, are important variables to consider when calculating the maximum number of dCas9 molecules that can be added to the flow cell. While the number of captured DNA copies was linear between 1- and 8-fold dCas9 molecules, the number of captured DNA copies did not quite double with the doubling of dCas9 molecules (slope=−0.797 Ct) giving evidence that layering the dCas9 molecules might lead to a slight suboptimal binding of cfDNA.

It was investigated if increasing the width of the flow channel from 0.5 mm to 5 mm would affect the flow rate required to achieve desired capture of the ctDNA (FIG. 2A). Similar to the flow cell with a 0.5 mm wide flow channel, the basic vortex-generating flow cell, after flowing 10 mL of plasma with 4,000 copies of spiked-in BRAF^{Mut} DNA at a range of flow rates between 6 and 104 mL/min for 20 minutes, it was found that the number of captured BRAF^{Mut} DNA copies increased with linearity between the flow rates of 6 and 78 mL/min (Slope=−0.474 Ct; R²=0.783; coefficient P<0.001) (FIG. 2B). Again, the optimal flow rate was 78 mL/min and, on average, 42 copies of BRAF^{Mut} DNA were captured. No evidence was found that the number of captured off-target ACTB DNA copies changed as the flow rate changed between the flow rates of 6 and 78 mL/min (Slope=0.075 Ct; R²=0.053; coefficient P=0.407) (FIG. 2C). The size of the flow channel, which affects the Reynolds number, had little to no effect on the flow rate required to achieve the optimal capture of ctDNA.

Modifications of the capture chamber were investigated to determine the effects of flow rate on the capture of the ctDNA. In the third, small capture chambers vortex-generating, flow cell, two capture chambers half the size of the capture chamber of the first vortex-generating flow cell, from 18 mm to 9 mm width were designed (FIG. 3A). After flowing 10 mL of plasma with 4,000 copies of spiked-in BRAF^{Mut} DNA at a range of flow rates between 3 and 26 mL/min for 20 minutes, the number of BRAF^{Mut} DNA copies captured increased as the flow rate increased with linearity between the flow rates of 3 and 12 mL/min (Slope=−0.757 Ct; R²=0.711; coefficient P=0.004) (FIG. 3B). At a flow rate of 12 mL/min 49 copies of BRAF^{Mut} DNA, on average, were captured. In this flow cell, the number of captured off-target ACTB DNA copies also increased as the flow rate increased between the flow rates of 3 and 12 mL/min (Slope=−0.656 Ct; R²=0.381; coefficient P=0.022) (FIG. 3C). From these results, the optimal capture rate of the ctDNA can be achieved with a lower flow rate (12 mL/min in comparison to 78 mL/min) when the size of the capture chamber is decreased by half.

A staggered herringbone mixer was used to determine the effects on the capture rate of ctDNA in plasma. Staggered herringbone mixers use repeated patterns of grooves to generate chaotic twisting flow and have been shown to be an efficient passive microfluidic mixer. This flow cell was designed to have a positive staggered herringbone mixer pattern and was used with forward flow (FIG. 4A), which has been shown to have high mixing efficiency.

In the positive staggered herringbone mixer flow cell, after flowing 10 mL of plasma with 4,000 copies of spiked-in BRAF^{Mut} DNA at a range of flow rates between 3 and 26 mL/min for 20 minutes, the number of BRAF^{Mut} DNA copies captured increased as the flow rate increased between the flow rates of 3 and 12 mL/min (Slope=−0.762 Ct; R²=0.711; coefficient P=0.002) (FIG. 4B). At a flow rate of 12 mL/min, 45 copies of BRAF^{Mut} DNA, on average, were captured. In this flow cell, the number of off-target ACTB DNA copies captured decreased as the flow rate increased between the flow rates of 3 and 26mL/min, although this trend was not statistically significant (Slope=0.391 Ct; R²=0.300; coefficient P=0.065) (FIG. 4C).

### Example 2

### Devices and Capture of ctDNA

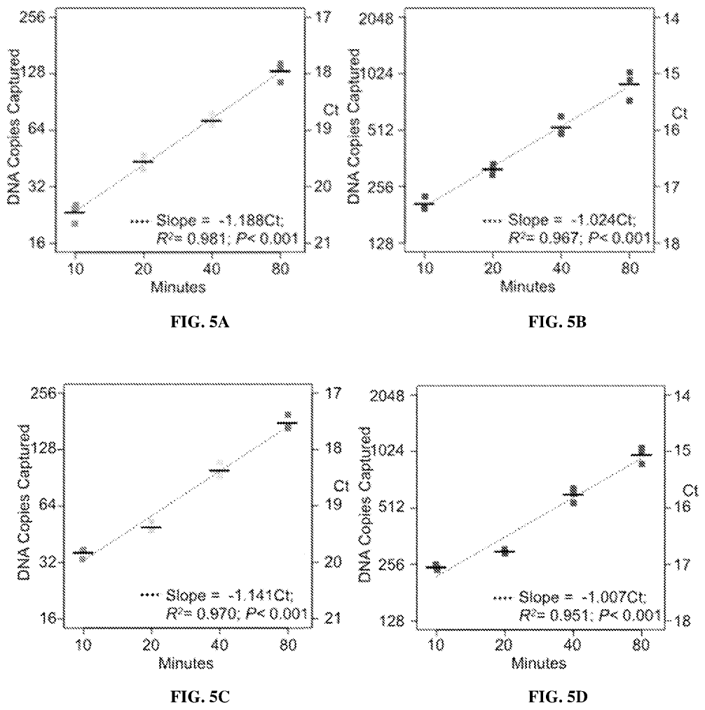

Since all four flow cells showed the ability to reach the desired capture rate of ctDNA in the unaltered flowing plasma, flow cells that use different fluid dynamic principles and optimal flow rates were investigated for showing capture BRAF^{Mut} DNA with similar linearity over time. For this comparison, the basic vortex-generating flow cell (FIG. 1A) with the optimal flow rate of 78 mL/min and the staggered herringbone mixer flow cell (FIG. 4A) with the optimal flow rate of 12 mL/min were selected. After flowing 10 mL of plasma with 4,000 spiked-in BRAF^{Mut} DNA copies for 10, 20, 40, or 80 minutes, in the vortex-generating flow cell, the number of BRAF^{Mut} DNA copies and off-target ACTB DNA copies doubled as the flow time doubled for both BRAF^{Mut} and off-target ACTB DNA (Slope=−1.188 Ct; R²=0.981; coefficient P<0.001) and (Slope=−1.024 Ct; R²=0.967; coefficient P<0.001), respectively (FIGS. 5A-5B). With the staggered herringbone mixer flow cell, the number of BRAF^{Mut} DNA copies and off-target ACTB DNA copies doubled as the flow time doubled (Slope=−1.141 Ct; R²=0.970; coefficient P<0.001) and (Slope=−1.007 Ct; R²=0.951; coefficient P<0.001) (FIGS. 5C-5D). After 20 minutes of flow time, there was no difference in the number of BRAF^{Mut} DNA copies captured between the basic vortex-generating flow cell and the SHM flow cell (t-test P=0.143).

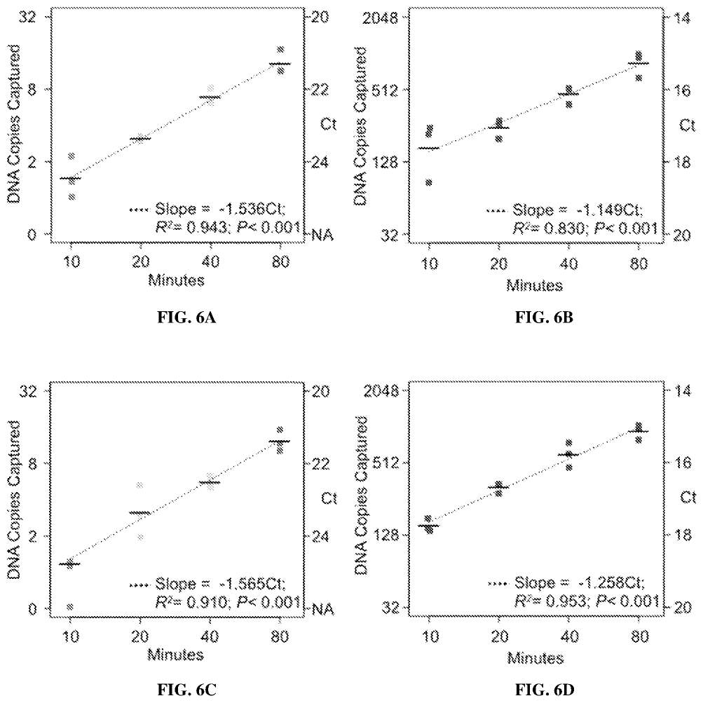

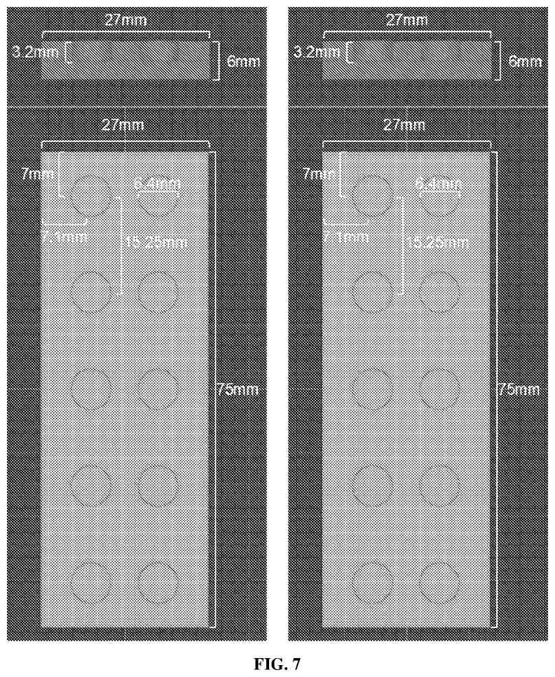

Finally, with the same two flow cells, it was investigated if the number of BRAF^{Mut} DNA copies spiked into the plasma affected the ability of dCas9 to capture cfDNA over time. Using 10 mL of plasma with 400 copies of BRAF^{Mut} DNA spiked into it, the unaltered plasma was flowed through the vortex-generating flow cell and staggered herringbone mixer flow cell for 10, 20, 40, and 80 minutes. Again, with the vortex-generating flow cell, the number of BRAF^{Mut} DNA copies and off-target ACTB DNA copies doubled as the flow time doubled (Slope=−1.536 Ct; R²=0.943; coefficient P<0.001) and (Slope=−1.149 Ct; R²=0.830; coefficient P<0.001) respectively (FIGS. 6A-6B). Similarly, with the staggered herringbone mixer, the number of BRAF^{Mut} DNA copies and off-target ACTB DNA copies doubled as the flow time doubled (Slope=−1.565 Ct; R²=0.910; coefficient P<0.001) and (Slope=−1.258 Ct; R²=0.953; coefficient P<0.001) respectively (FIGS. 6C-6D). After 20 minutes of flow time, the number of BRAF^{Mut} DNA copies captured was similar between the basic vortex-generating flow cell and the SHM flow cell (t-test P=0.989).

The rate of BRAF^{Mut} DNA copies and off-target ACTB DNA copies increased with linearity over time and was similar between the basic vortex-generating flow cell (with the flow rate of 78 mL/min) and the SHM flow cell (with the flow rate of 12 mL/min). The rate and number of captured DNA copies were similar between these two flow cells after flowing 10 ml of plasma with either 400 or 4,000 copies of spiked in BRAF^{Mut} DNA at a range of flow times. Together, these results suggested that the mass transfer of ctDNA to the dCas9, the kinetics and equilibrium of the dCas9, number of dCas9 molecules, and flow time are the four major variables that affect the ability of the dCas9 capture system to capture ctDNA in flowing unaltered plasma.

It is understood that the foregoing detailed description and accompanying examples are merely illustrative and are not to be taken as limitations upon the scope of the disclosure, which is defined solely by the appended claims and their equivalents. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A device comprising:
a sample inlet;
a sample outlet;
a channel fluidically connecting the sample inlet and sample outlet, wherein the channel comprises at least one mixing element; and
a plurality of capture complexes each capture complex comprising an RNA-guided DNA binding protein, or a functional fragment thereof, bound to a guide RNA configured to at least partially hybridize to a cfDNA of interest.

2. The device of claim 1, wherein the plurality of capture complexes are immobilized on one or more interior surfaces of the channel.

3. The device of claim 1 or 2, wherein the channel is substantially linear.

4. The device of any of claims 1-3, wherein the at least one mixing element comprises one or more chambers spanning the width of the channel and expanding out from the channel.

5. The device of claim 4, wherein the one or more chambers expand out perpendicularly from direction of the channel.

6. The device of claim 4 or 5, wherein the plurality of capture complexes are immobilized on one or more interior surfaces of the one or more chambers.

7. The device of any of claims 1-6, wherein the at least one mixing element comprises a plurality of microstructures.

8. The device of claim 7, wherein the microstructures include ridges, channels, protrusions, or any combination thereof.

9. The device of any of claims 1-8, wherein the at least one mixing element has a height of between 30-80% of height of the channel.

10. The device of any of claims 1-9, wherein the plurality of capture complexes are linked to magnetic particles.

11. The device of claim 10, wherein the plurality of capture complexes are linked by a biotin streptavidin linker.

12. The device of claim 11, wherein the RNA-guided DNA binding protein further comprises a biotin tag.

13. The device of any of claims 10-12, wherein the plurality of capture complexes are magnetically immobilized to the one or more locations on the interior surfaces of the channel.

14. The device of any of claims 1-13, wherein the device further comprises one or more magnets.

15. The device of any of claims 1-14, wherein the plurality of capture complexes are immobilized through immunoprecipitation.

16. The device of any of claims 1-15, wherein the RNA-guided DNA binding protein is a CRISPR-associated (Cas) protein.

17. The device of any of claims 1-16, wherein the RNA-guided DNA binding protein is Cas9.

18. The device of claim 17, wherein the Cas9 is catalytically inactivated.

19. The device of any of claims 1-18, wherein the plurality of cfDNA complexes comprises more than one type of cfDNA complex each type having a guide RNA configured to at least partially hybridize to a different cfDNA of interest.

20. A system comprising:
a plurality of RNA-guided DNA binding proteins, or a functional fragment thereof;
one or more guide RNAs configured to at least partially hybridize to a cfDNA of interest; and
a device comprising:
a sample inlet;
a sample outlet; and
a channel fluidically connecting the sample inlet and sample outlet, wherein the channel comprises at least one mixing element,
wherein the RNA-guided DNA binding proteins are configured for immobilization on one or more interior surfaces of the channel.

21. The system of claim 20, wherein the channel is substantially linear.

22. The system of claim 20 or 21, wherein the at least one mixing element comprises one or more chambers spanning the width of the channel and expanding out from the channel.

23. The system of any of claims 20-22, wherein the at least one mixing element comprises a plurality of microstructures.

24. The system of any of claims 20-23, wherein the microstructures include ridges, channels, protrusions, or any combination thereof.

25. The system of any of claims 20-24, wherein the at least one mixing element has a height of between 30-80% of height of the channel.

26. The system of any of claims 20-25, wherein the plurality of RNA-guided DNA binding proteins are linked to magnetic particles.

27. The system of claim 26, wherein the plurality of RNA-guided DNA binding proteins are linked by a biotin streptavidin linker.

28. The system of claim 26 or 27, wherein the RNA-guided DNA binding protein further comprises a biotin tag.

29. The system of any of claims 20-28, wherein the plurality of RNA-guided DNA binding proteins are magnetically immobilized to the one or more locations on the interior surfaces of the channel.

30. The system of any of claims 20-29, wherein the one or more guide RNAs are bound to the plurality of RNA-guided DNA binding proteins to form a capture complex.

31. The system of any of claims 20-30, wherein the RNA-guided DNA binding protein is a CRISPR-associated (Cas) protein.

32. The system of any of claims 20-31, wherein the RNA-guided DNA binding protein is Cas9.

33. The system of claim 32, wherein the Cas9 is catalytically inactivated.

34. The system of any of claims 20-33, further comprising one or more magnets.

35. The system of any of claims 20-34, further comprising a sample.

36. The system of claim 35, wherein the sample comprises blood or blood components.

37. The system of claim 36, wherein the blood component comprises plasma.

38. A method of capturing circulating free DNA (cfDNA) from a sample comprising introducing the sample into a device of any of claims 1-19 to form a complex comprising the capture complex and the circulating free DNA of interest.

39. The method of claim 38, wherein the sample is introduced at a flow rate of 3-120 mL/min.

40. The method of claim 38 or 39, wherein the mixing element comprises two chambers along a single portion of the channel, perpendicular to sample flow direction in the channel, and the flow rate is 70-120 mL/min.

41. The method of claim 38 or 39, wherein the mixing element comprises two chambers along different portions of the channel, perpendicular to sample flow direction in the channel, and the flow rate is 10-30 mL/min.

42. The method of claim 38 or 39, wherein the mixing element comprises a plurality of microstructures and the flow rate is 10-20 mL/min.

43. The method of any of claims 38-42, wherein the sample is a biological sample

44. The method of any of claims 38-43, wherein the biological sample comprises blood or blood components.

45. The method of claim 44, wherein the blood component comprises plasma.

46. The method of any of claims 38-45, wherein the circulating free DNA is of microbial or viral origin.

47. The method of any of claims 38-45, wherein the circulating free DNA is circulating tumor DNA.

48. The method of any of claims 38-47, further comprising removing remaining sample from the device.

49. The method of any of claims 43-48, further comprising analyzing at least a portion of the biological sample or the remaining biological sample for the presence or absence of at least one biomarker.

50. The method of any of claims 43-49, further comprising returning at least a portion of the remaining biological sample to the subject.

51. The method of any of claims 38-50, further comprising one or more of: purifying the cfDNA of interest; amplifying the cfDNA of interest; and sequencing the cfDNA of interest.

52. The method of any of claims 38-51, further comprising immobilizing the plurality of capture complexes in the device.

53. A kit comprising at least one device as in claims 1-19 or system as in claims 20-37.

54. The kit of claim 53, comprising two or more devices or systems, wherein each device or system comprises one or more gRNAs configured to hybridize to two or more different cfDNA of interest.
